(19) United States
(12) Patent Application Publication
(10) Pub. No.: US 2002/0037871 A1
(43) Pub. Date: Mar. 28, 2002

(54) Pharmaceutical use of adenosine agonists

(76) Inventors: Pnina Fishman, Herzliya (IL); Ilan Cohn, Herzliya (IL)

Correspondence Address: BIRCH STEWART KOLASCH & BIRCH, PO BOX 747, FALLS CHURCH, VA, 22040-0747, US

(21) Appl. No.: 09/871,963

(22) Filed: Jun. 4, 2001

Related U.S. Application Data
(63) Continuation-in-part of application No. 09/782,259, filed on Feb. 14, 2001.
Application No. 09/782,259 is a continuation-in-part of application No. 09/700,744, filed on Jan. 9, 2001.
Application No. 09/700,744 is a continuation-in-part of application No. PCT/IL00/00014, filed on Jan. 7, 2000.

(30) Foreign Application Priority Data
Jan. 7, 1999 (IL) 127947

(51) Int. Cl. A61K 31/7076

(52) U.S. Cl. 514/46

(57) ABSTRACT

The present invention relates to a method for inducing proliferation of the hematopoietic system, in particular, of bone marrow cells, comprising administering to a subject an effective amount of an adenosine A1 receptor agonist. The method of the invention may be utilized n a variety of clinical situations where such proliferation is therapeutically beneficial.

The active ingredient within the pharmaceutical composition of the invention may be a compound of general formula (I) as described herein, or any other compound or substance which specifically binds to and/or activates the A1 adenosine receptor and acts as an agonist to the receptor's natural ligand.

### FIELD OF THE INVENTION

This invention relates to drugs for use in cancer therapy. More specifically, the present invention concerns drugs which induce proliferation of cells of the hematopoietic system.

### PRIOR ART

The following is a list of prior art references considered to be relevant as background to the invention:

1. Daly, J. W., J: Med Chem., 25:197-207, 1982.

2. Stiles, G. L., Clin Res., 38:10-18, 1990.

3. Collis, M. G., Pharmacol Ther, 41:143-162, 1989.

4. Fishman et al., Proceeding of the American Association for Cancer Research, 39:470, 1998.

5. Moos, W. H., et a.,, J Medicinal Chemistry 28:1383-1384, 1985.

6. Jacobson, K. A. et a.,, J Medicinal Chemistry 28:1341-1346, 1985.

7. U.S. Pat. No. 5,998,387.

8. U.S. Pat. No. 5,998,399

9. U.S. Pat. No. 5,499,605.

10. U.S. Pat. No. 4,791,103.

11. Snowdy et al British J. Pharmacol. 126:137-146 (1999)

12. WO 98/08855

13. WO 00/71558

### BACKGROUND OF THE INVENTION

Adenosine is an extracellular messenger generated by all cells in the body. It is known to regulate different physiological processes within cells through binding to specific cell surface receptors—A1 and A2 receptors ^{(1,2,3)}. It was recently demonstrated that adenosine inhibits proliferation of tumor cells and induces proliferation of bone marrow cells ⁽⁴⁾. Further more it was also shown that adenosine can protect white blood cells, particularly neutrophils, from destruction which is otherwise caused by chemotherapeutic drugs ⁽⁴⁾.

### SUMMARY OF THE INVENTION

The present invention is based on die surprising findings that (i) the effect of adenosine in inducing proliferation of bone marrow cells can be inhibited by A1 receptor antagonists (antagonist tat inhibits binding of adenosine to adenosine A1 receptor), and (ii) die effect of adenosine can be mimicked by an adenosine A1 receptor agonist (“A1RAg”). These findings led to the conclusion That the bone marrow proliferation-induction effect of adenosine is mediated, at least to some extent through the A1 receptor, and that accordingly A1RAg way be used to induce proliferation of bone marrow cells, in a wide variety of clinical situations where such proliferation is therapeutically beneficial.

The present invention provides, by a first of its aspects, a method for inducing proliferation of bone marrow cells, comprising administering to a subject in need of such treatment a therapeutically effective amount an A1RAg.

According to one preferred embodiment of this aspect, the subject in need of said treatment has a low white blood cell count, preferably as a result of a cancer disease, whereby the white blood cell level is reduced as a results, for example of chemotherapy or radiotherapy treatment.

By a second of its aspects, the present invention provides a pharmaceutical composition for use in inducing proliferation of bone marrow cells, comprising a pharmaceutically acceptable carrier, excipient or diluent and, as an active ingredient, an effective amount of an A1RAg.

The present invention provides, by a third of its aspects, use of an A1RAg for the production of a pharmaceutical composition for use in inducing proliferation of bone marrow cells.

The term “effective amount” used above and below should be understood as meaning an amount of an A1RAg which is capable of achieving a desired therapeutic effect, particularly, in inducing proliferation of bone marrow cells. The desired therapeutic effect depends on the type and mode of treatment. When, for example, said A1RAg is administered to counter drug-induced leukopenia, an effective amount thereof may be an amount which protects the treated subject against the drug-induced reduction in the count of leukocytes, particularly neutrophils; an amount of the active ingredient which can give rise to an increase in an already decreased level of such cells, e.g. restore the level to a normal level or sometimes even above; etc. The man of the art will have no difficulties, on the basis of a limited number of routine experiments, to determine an effective amount in each case.

As will be appreciated, the effective amount may also depend on the treated subject's gender, on the individual's weight, on the therapeutic regime, namely whether the A1RAg is administered once daily, several times daily, once in several days, etc. Furthermore, the effective amount may depend on the exact nature or etiology of the disease or condition which is being treated or intended to be prevented.

According to one embodiment of the invention, the A1RAg are adenosine derivatives carrying at least an N⁶-substituent, Other positions may also be substituted. In fact, it has been found that the biological activity of an adenosine derivative may be enhanced by modifying other parts of the nucleotide, for example, at the 2- and/or 5′-positions (e.g. with chloro atoms). Such substituents were found to increase tie molecule's A1 selectivity.

The adenosine derivatives which can be used according to the present invention are generally defined by the following formula (I):—

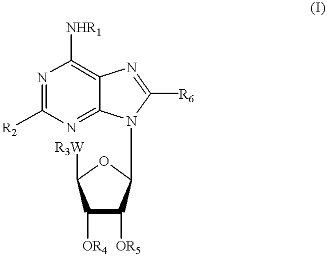

wherein

R₁ represents a lower alkyl, substituted or unsubstituted cycloalkyl; a hydroxyl or hydroxyalkyl; a phenyl, lower alkyl phenyl or anilide, all optionally substituted by one or more substituents; —SOR, —SO₂R^{c}, —SO₃H, —SO₂NR^{a}R^{b}, —OR^{a}, —SR,^{a}, —NHSO₂R^{c}, —NHCOR^{a}, —NR¹R^{b}, or —NHR^{a}CO₂R^{b}; wherein

R^{a} and R^{b} represent independently a hydrogen, lower alkyl, alkanoyl, amine, phenyl or naphthyl, the alkyl group optionally being substituted with a substituted or unsubstituted phenyl or phenoxy group; or when R₁ represents —NR^{a}R^{b}, said R^{a} and R^{b} form together with tie nitrogen atom a 5- or 6- memebered heterocyclic ring optionally containing a second heteroatom selected from oxygen or nitrogen, which second nitrogen heteroatom may optionally be firer substituted by hydrogen or lower alkyl; or —NR^{a}R^{b} is a group of general formulae (II) or (III):—

[chemical structure]

wherein

n is an integer from 1to 4;

Z is hydrogen, lower alkyl or hydroxyl;

Y is hydrogen,, lower alkyl, or OR′ where R′ is hydrogen, lower alkyl or lower alkanoyl;

A, is a bond or a lower alkylene;

X and X′ are each independently hydrogen, lower alkyl, lower alkoxy, hydroxy, lower alkanoyl, nitro, haloalkyl such as trifluoromethyl, halogen, amino, mono- or di-lower alkyl amino, or when X and X′ are taken together a methylenedioxy group;

R^{c} represents a lower alkyl;

R₂ represents hydrogen; halogen; substituted or unsubstituted lower alkyl or alkenyl, group; lower haloalkyl or alkenyl cyano; acetoamido; lower alkoxy; lower alkylamino; NR^{d}R^{e} where R^{d} and R^{c} are independently hydrogen, lower alkyl, phenyl or phenyl substituted by lower all, lower alkoxy, halogen or haloalkyl or alkoxyl; —SR^{f} where R^{f} is hydrogen, lower alkyl, lower alkanoyl, benzoyl or phenyl;

W represents the group —OCH₂—, —NHCH₂—, —SCH₂— or —NH(C═O)—;

R₃, R₄ and R₅ represent independently a hydrogen, lower alkyl or lower alkenyl, branched or unbranched C₁-C₁₂ alkanoyl, benzoyl or benzoyl substituted by lower alkyl, lower alkoxy, halogen, or R₄ and R₅ form together a 5-membered ring optionally substituted by a lower alkyl or alkenyl; R₃ further represents independently a phosphate, hydrogen or dihydrogen phosphate, or an alkali metal or ammonium or dialkali or diammonium said thereof;

R₆ represents a hydrogen or halogen atom; or

one of the substituents R₁ to R₆ is a sulfohydrocarbon radical of the formula —R^{b}— SO₃—R^{g}, wherein R^{g} represents a group selected from C₁-C₁₀ aliphatic, phenyl and lower alkyl substituted aromatic group which may be substituted or unsubstituted and R^{h} represents a monovalent cation, and the non-sulfur containing substituents being as defined above. The remaining R groups being a hydrogen or halogen atom, an unsubstituted hydrocarbon or any other non-sulfur containing group as defined above.

Suitable monovalent cations include lithium, sodium, potassium, ammonium or trialkyl ammonium, which will enable dissociation to take place under physiological conditions.

The active ingredient may be as defined above or in the form of salts or solvates thereof, in particular physiologically acceptable salts and solvates thereof Further, when containing one or more asymmetric carbon atoms, the active ingredient may include isomers and diastereoisomers of the compounds of formula (I) and mixtures thereof.

The hydrocarbon chains used herein may include straight or branched chains. In particular, the terms “alkyl”, “alkenyl” or “alkynyl” as used herein mean a straight or branched chain alkyl or alkenyl groups.

The terms “lower” used in connection with the definition of carbohydrates refer to such a carbohydrate containing up to 10 carbon atoms and preferably up to 6 carbon atomes.

Preferred adenosine derivatives of formula (I) are the N⁶-cyclopentyl adenosine (CPA), 2-chloro-CPA (CCPA), and N⁶-cyclohexyl adenosine (CHA) derivatives, the preparation of which is well known to the person skilled in the art. Other adenosine derivatives which are known to be selective to the A1 receptor are those wherein R₁ is a anilide group, the latter may be unsubstituted or substituted for example with hydroxyl, alkyl, alkoxy or with a group —CH₂C(O)R″, R″ being an hydroxyl group —NHCH₃, —NHCH₂CO₂C₂H₅,(ethyl glycinate), tuloidide (also in which the methyl moiety is replaced with a haloalkyl moiety), or with a group —CH₂C(O)NHC₆H₂C(O)R′″, in which R′″ represents a group to yield a methyl ester substituent (—OCH₃), an amide substituent (e.g. R′″ being a group —NHCH₃), or R′″ being a hydride, ethylenediamine, —NHC₂H₅NHC(O)CH₃, 4-(hydroxyphenyl)propionyl, biotinylated ethylene diamine or any other suitable hydrocarbon which renders the compound an A1 agonist. The preparation of some of the above specific adenosine derivatives is described in the art ⁽⁵⁻⁸⁾.

Alternatively, the N⁶-substituted adenosine derivatives used as active ingredients according to the present invention may be those containing an epoxide moiety and more particularly are a cycloalkyl epoxy containing adenosine derivative (e.g. oxabicyclo such as norbornanyl or oxatricyclo such as adamantanyl). Some such compounds may be defined by general formula (I),

wherein R₁ is a group of general formulae (IVa) and (IVb):—

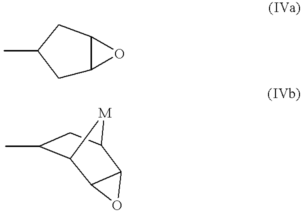

wherein M is a lower alkyl group as defined above.

Embodiments of the agonist compounds having an epoxide N⁶-norbornyl group include the endo and exo isomers and more particularly, can be one of four isomers: the 2R-exo, 2R-endo, 2S-exo and 2S-endo form.

Another embodiment of the N⁶-norbornyl derivative may include an oxygen atom at the N¹-position of the purine ring, This compound is known by die name N⁶-(5,6-epoxynorborn-2-yl) adenosine-1-oxide.

Yet further, the N⁶-substituted adenosine derivatives used as active ingredients according to the present invention may be those of the formula (I) in which R₁ represents a monocyclic or polycyclic heterocyclic group consisting of from 3 to 15 atoms, at least one of which is selected from the group consisting of N, O, P and S—(O)₀₋₂; the heterocyclic group optionally substituted by one or more substituents selected from the group consisting of halo, oxo, hydroxyl, lower alkyl, substituted lower alkyl, alkoxy, aryl, acyl, aryloxy, carboxyl, substituted aryl heterocycle, heteroaryl, substituted heteroaryl, cycloalyl, substituted cycloalkyl, —NO₂, and —CN.

In this preferred embodiment, the substituents R₄ and R₅ may form together a 5-membered ring optionally substituted by a lower alkyl or alkenyl, and in addition R₃, R₄ and R₅ may represent each a group of the formula R₇C(O)— wherein R₇ may be the same or different and is selected from the group consisting of C₁₋₁₅ alkenyl, C₂₋₁₅ alkenyl, C₂₁₅ alkynyl, heterocyclyl, aryl, and heteroaryl, or which alkyl, alkenyl, alkynyl, aryl, heterocyclyl and heteroaryl are optionally substituted with 1 to 3 substituents independently selected from the group of halo, alkyl —NO₂, heterocyclyl, aryl, heteroaryl, —CF₃, —CN, —OR²⁰, —SR²⁰, —N(R²⁰)₂, —S(O)R²², —SO₂R²², —SO₂N(R²⁰)₂, —SO₂NR²⁰C(O)R²², —SO₂NR²⁰CO₂R²², —SO₂NR²⁰C(O)N(R²⁰)₂, —N(R²⁰)₂, —NC(O)R²², —NR²⁰C(O)R²², —NR²⁰CO₂R²², —NR²⁰C(O)N(R²⁰)₂, —NR²⁰C(NR²⁰)NHR²³, —C(O)R²⁰, —CO₂R²⁰, —CON(R²⁰)₂, —CONR²⁰SO₂R²², —NR²⁰SO₂R²², —SO₂NR²⁰CO₂R²², —OC(O)NR²⁰SO₂R²², —OC(O)R²⁰, —C(O)OCH₂OC(O)R²⁰, and —OC(O)N(R²⁰)₂; wherein

R²⁰ is a member selected from the group consisting of C₁₋₁₅ alkyl, C₂₋₁₅ alkynyl, C₂₋₁₅ alkynyl, heterocyclyl, aryl and heteroaryl, which alkyl, alkenyl, alkynyl, heterocyclyl, aryl and heteroaryl are optionally substituted with 1 to 3 substituents independently selected from halo, alkyl, mono or dialkylamino, alkyl-, aryl- or heteroaryl amide, CN, —OC₁₋₆ alkyl, —CF₃, aryl and heteroaryl; and

R²² is a member selected from the group consisting of C₁₋₁₅ alkyl, C₂₋₁₅ alkynyl, C₂₋₁₅ alkynyl, heterocyclyl, aryl and heteroaryl, which alkyl, alkenyl, alkynyl, heterocyclyl, aryl and heteroaryl are optionally substituted wit 1 to 3 substituents independently selected from halo, alkyl, mono or dialkylamino, alkyl-, aryl- or heteroaryl amide, CN, —OC₁₋₆ alkyl, —CF₃, aryl and heteroaryl.

Specific derivatives according to this embodiment include, without being limited thereto, compounds in which R₁ is selected from the group consisting of 3-tetrahydrofuranyl, 3-tetrahydrothiofuranyl, 4-pyranyl and 4-thiopyranyl. The preparation and biological function of some such derivatives has already been described⁽¹¹⁻¹³⁾. A preferred adenosine derivative according to the present embodiment includes is N-(3(R)-tetrahydrofuranyl)-6-aminopurine riboside⁽¹¹⁾.

Phaaceutically acceptable salts of the compound of general formula (I) include those derived from pharmaceutically acceptable inorganic and organic acids. Examples of suitable acids include hydrochloric, hydrobromic, sulphoric, nitric, perchloric, fumaric, maleic, phosphoric, glycollic, lactic, salicylic, succinic, toluene-p-sulfonic, tartaric, acetic, citric, methanesulfonic, formic, benzoic, malonic, naphthalene-2-sulfonic and benzenesulfonic acids.

It should be noted that, at times, the active ingredient may be an adenine s derivative in which the β-D-ribofuranozyl moiety of the adenosine is replaced with a hydrogen or phenyl group.

The invention has a wide range of therapeutic utilities and provides treatment for a wide range of diseases, disorders or conditions in both human and non-human animals, where induction of proliferation of bone marrow cells may be beneficial to the treated subject. Therapeutic applications include immunomodulation in a subject having a weak immune system, for example: as a result of a genetic disorder; as a result of an infection by an infectious agent, e.g. a virus; as a result of a general stress situation, e.g. following a car or another accident, etc.; as a result of a treatment which causes reduction in the level of leukocytes, particularly neutrophils, e.g. a chemotherapy or treatment with a neuroleptic drug; etc.

A treatment according to the invention may be used to reduce the risk of infection resulting from congenital or acquired neutropenias.

The present invention may also be used for the treatment of subjects having a low count of white blood cells, either a general low count or a count of a specific class of white blood cells, e.g. neutrophils. A weakened immune system manifested by a reduction in white blood cell count, is often seen in cancer patients, and when this occurs, this may have a severe effect on the treated patent, and may at times even be a cause of death. In such a ease it is thus import to try and increase the white blood cell count. This may be achieved by the treatment in accordance with the invention.

Reduction of white blood cell count, particularly of neutrophils, is very often an undesired side effect of a variety of treatments, including: anti-cancer therapy by chemotherapy or radiotherapy; treatment of a subject with neuroleptic drugs; etc. The active ingredient of the invention may be used in such subject to counter these undesired side effects of the treatment In accordance with some therapeutic regimes, the active ingredient of the invention may be administered prior to such treatment or concurrently therewith. For example, in the case of a treatment with a chemotherapeutic drug or treatment with a neuroleptic drug, the active ingredient of the invention may be administered either prior to the onset of treatment wit the chemotherapeutic or the neuroleptic drug during such treatment, or may also at times be given after such treatment In other words, the active ingredient of the invention may be used either as a preventive agent, namely to prevent reduction of the white blood cell level as a result of the treatment, or may be used as an acute therapeutic agent for simulating an increase in the level of the white blood cells after the level was reduced as a result of said treatment.

In accordance with one embodiment of the invention, an anti-cancer chemotherapeutic agent or a neuroleptic drug may be combined in one formulation is with the active ingredient of the invention.

### BRIEF DESCRIPTION OF THE FIGURES

In order to understand the invention, and to see bow it may be carried out in practice, a preferred embodiment will now be described, by way of non-limiting example only, wit reference to the accompanying drawings, in which:

FIG. 1 is a bar graph showing results of an in vitro assay in which proliferation of bone marrow cells was tested without adenosine (dense stripes) and with adenosine (spaced stripes) together with adenosine A1 receptor antagonist (DPCPX) and adenosine A2 receptor antagonist (DMPX) as compared to a control without any additional added drug. The bar graph shows results of a [³H]thymidine incorporation assay.

FIG. 2 shows [³H]thymidine incorporation assay of a control bone marrow cell preparation (“control”), in the presence of adenosine (“control+adenosine”) and in the presence of two different concentrations of an A1 receptor agonist, (“CPA”).

### DETAILED DESCRIPTION OF A PREFERRED EMBODIMENT

The invention will now be illustrated by the following description of some experiments carried out according thereto.

### EXAMPLE 1

### In Vitro Evaluation of A1RAg

Materials And Methods

Mice

Female ICR or C57BL/61 mice aged 3 months, weighing an average of 25 g were used. The mice were purchased from Harlan Laboratories, Jerusalem, Israel. Standardized pelleted diet and tap water were supplied.

All drugs were purchased from Sigma Chemical Co. St. Louis, Mo. Adenosine was dissolved in water and kept as a stock solution in a concentration of 1 mM. For in vitro studies, dilutions in RPMI medium were carried out and final concentrations of 100, 50, 25, 10 and 5 μM were used. For in vivo studies, the stock solution was diluted with PBS to a concentration of 3 mM and 0.5 ml was injected intraperitoneally to mice. 1,3-dipropyl-8-cyclopentylxanthine (DPCPX), an adenosine A1 receptor antagonist, 3,7-dimethyl-1-propargyl-xantane (DMPX) an A2 receptor antagonist and N-cyclopentyladenosine (CPA), a selective A1 receptor agonist were added to a culture of proliferating bone marrow cells.

Evaluation of Bone Marrow Cell Proliferation in vitro

Bone marrow cells were obtained from the femur of C57BJ,/6J mice. Cells were disaggregated by passing through a 25 G needle. [³H]-Thymidine incorporation assay was used to evaluate the proliferative capability of the bone marrow cells.

Cells (3×10⁴ /well) were incubated with RPMI medium, containing 10% fetal calf serum (FCS) (Biological Industries, Beit Haemek, Israel) and adenosine, adenosine antagonists or the agonist in 96 microtiter plates for 48 h. Cultures containing cells were suspended in RPMI medium and 10% FCS served as controls. In the last 6 hours of incubation, each well was pulsed with 1 μCi [³H]-thymidine. Cells were harvested and the ³[H]-thymidine uptake was determined in an LKB liquid scintillation counter (LKB, Piscataway, N.J., USA).

Results

Effect of Adenosine, Adenosine Receptor Antagonists and Agonist on Bone Marrow Cell Proliferation

Exposure of bone marrow cells to adenosine at concentrations of 10-50 μM stimulated ³[H]-thymidine incorporation in a concentration dependent manner (Fishman et at ⁽⁴⁾).

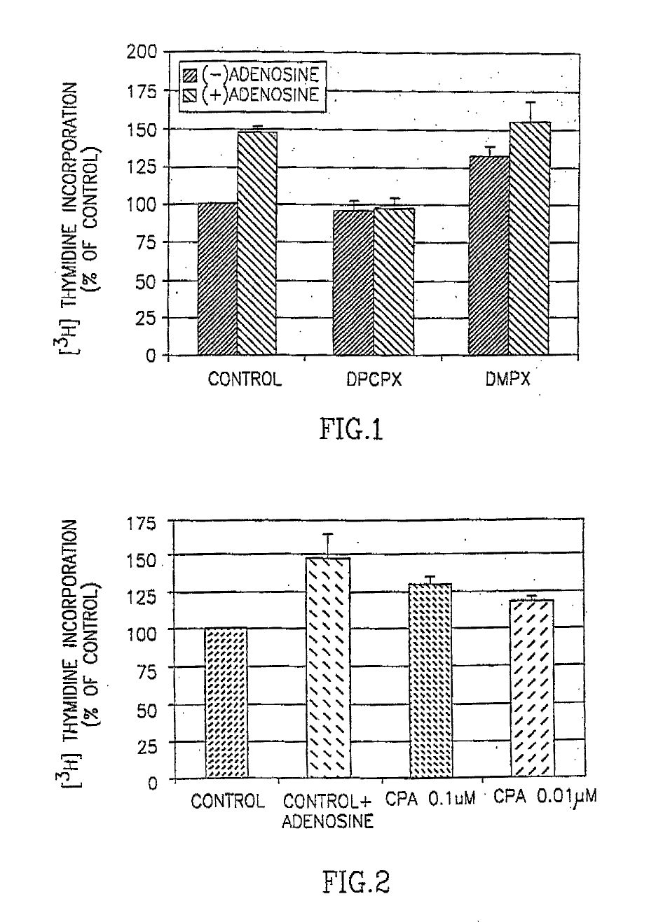

To evaluate which adenosine receptor is responsible for this stimulatory effect, two adenosine receptor antagonists were used, i.e., DPCPX (A1 antagonist) and DMPX (A2 antagonist). The effect of the antagonists on bone marrow cell proliferation was examined with and without adenosine. In the absence of adenosine, the effect of endogenous adenosine which is released by the bone marrow cells and affects the same cells by a paracrine way, was evaluated, DPCPX (0.1 μM which block the A1 receptor, significantly reversed the stimulatory effect of adenosine on bone marrow cell proliferation. DMPX (0.1 μM, given without or with adenosine induced a stimulatory effect on bone marrow cell proliferation (FIG. 1). These results show that the A1 receptor is responsible for the stimulatory effect of adenosine. To confirm this result CPA, a selective adenosine A1 receptor agonist was added to a culture of bone marrow cells. CPA induced a statistically significant stimulation of bone marrow cell proliferation at concentrations of 0.1 and 0.01 μM (FIG. 2).

### EXAMPLE 1

### In Vivo Evaluation of A1RAg

Materials And Methods

Mice

Male ICR mice of the age of two months and weighing an average of 25 gr were employed, The mice were purchased from Harlan Laboratories, Jerusalem,, ISRAEL.

Drug

10 The cyclopentyl adenosine (CPA), an A1 adenosine receptor agonist, was used. This drug was purchased Sigma Chemical Co. (St. Louis Mo., USA)

In Vivo Evaluation

Protection of CPA-treated mice against myelotoxic effects of chemotherapy was evaluated. To this end, mice were provided with a chemotherapeutic drug, a combination of the drug with CPA or with a vehicle only. The drug of choice was cyclophosphamide (CPY, given by intraperinoeal injection),

In particular, three test groups of mice were treated daily according to the following regimen:

1. Control group—vehicle only.

2. Chemotherapy—CYP (50 mg/kg body weight).

3. Chemotherapy+chemoprotection—CYP (50 mg/kg body weight)+CPA (6 μg/kg body weight, given through per os administration, 48 h and 72 h following chemotherapy treatment).

To test the myeloprotective effect of CPA, blood samples were withdrawn from the above-mentioned groups 96 h, 120 h and 144 h following treatment with CYP, the following measurements were taken.

Blood cell count-Blood cell count was carried out in a Coulter counter and s differential cell counts were performed on smear preparations stained with May-Grunvald-Giemsa solution. Serum was separated from the whole blood and was kept at −70° C.

G-CSF level- granulocyte colony stimulating factor (G-CSF) level in the serum was determined by the use of a commercial ELISA kit (R&D Systems). G-CSF activity was expressed in pgr/ml according to a standard curve of recombinant G-CSF supplied by R,&D Systems.

Results

The results obtained show that CPA, an A1RAg provides in vivo protection against myelotoxicity of chemotherapeutic drugs. Table 1 provides the levels of G-CSF, 15 the number of white blood cells (WBC) and the number of neutrophils form the treated mice, as determined after 96 hours, 120 hours and 144 hours post treatment with the chemotherapeutic drug. Control levels were 6100 WBC and 1200 Neutrophils.

TABLE 1

| | | | Neutrophils |
|---|---|---|---|
| Treatment | G-CSF (pgr/ml) | WBC (no.) | (no.) |
| CYP (96 hrs) | 511.30 | 2620 | 377 |
| CYP (96 hrs) + CPA | 1508.29 | 1875 | 487 |
| CYP (120 hrs) | 333.03 | 2800 | 490 |
| CYP (120 hrs) + CPA | 887.65 | 4260 | 494 |
| CYP (144 hrs) | 871.14 | 3900 | 1326 |
| CYP (144 hrs) + CPA | 811.72 | 5289 | 2089 |

These results show that treatment with chemotherapeutic drug only reduce significantly die number of leukocytes and neutrophils while with the combined treatment (CYP+CPA) the level of G-CSF is higher than that obtained with CYP only and the numbers of leukocytes and neutrophils were increased.

Thus, it may be concluded that CPA, given orally to mice, induces in vivo G-CSF production and acts as a chemoprotective agent.

1. A method of inducing proliferation of bone marrow cells in a subject comprising administering to the subject an effective amount of an adenosine A1 receptor agonist.

2. A method according to claim 1, comprising administering the adenosine A1 receptor agonist to a subject having a low white blood cell count.

3. A method according to claim 1, wherein the treated subject is a cancer patient.

4. A method according to claim 3, wherein the treated subject is a cancer patient whose white blood cells level was reduced as a result of chemotherapy or radiotherapy.

5. A method according to claim 1, wherein said adenosine A1 receptor agonist is a compound of general formula (I):— 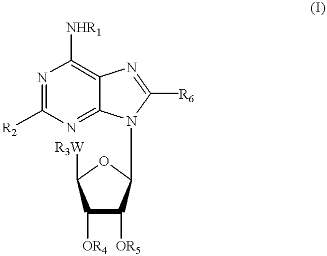
wherein
R₁ represents a lower alkyl, substituted or unsubstituted cycloalkyl; a hydroxyl or hydroxyalkyl; a phenyl, lower alkyl phenyl or anilide, all optionally substituted by one or more substituents; SOR^{c}, —SO₂R^{c}, —SO₃H, —SO₂NR^{a}R^{b}, —OR^{a}, —SR^{a}, —NHSO₂R^{c}, —NHCOR^{a}, —NR^{a}R^{b}, or —NHR^{a}CO₂R^{b}; wherein
R^{a} and R^{b} represent independently a hydrogen, lower alkyl, alkanoyl, amine, phenyl or naphthyl, the alkyl group optionally being substituted with a substituted or unsubstituted phenyl or phenoxy group; or when R₁ represents —NR^{a}R^{b}, said R^{a} and R^{b} form together with the nitrogen atom a 5- or 6-membered heterocyclic ring optionally containing a second heteroatom selected from oxygen or nitrogen, which second nitrogen heteroatom may optionally be further substituted by hydrogen or lower alkyl; or —NR^{a}R^{b} is a group of general formulae (II) or (III):— [chemical structure]
wherein.
n is an integer from 1 to 4;
Z is hydrogen lower alkyl or hydroxyl;
Y is hydrogen, lower alkyl, or —OR′ where R′ is hydrogen, lower alkyl or lower alkanoyl;
A is a bond or a lower alkylene;
X and X′ are each independently hydrogen, lower alkyl, lower alkoxy, hydroxy, lower alkanoyl, nitro, haloalkyl such as trifluoromethyl, halogen, ammo, mono- or di-lower alkyl amino, or when X and X′ are taken together a methylenedioxy group;
R^{c} represents a lower alkyl;
or
R₁ represents an epoxide substituent of general formulae (IVa) or (IVb): 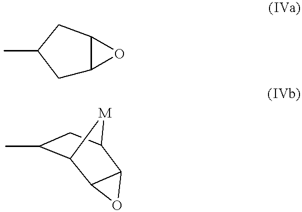
wherein M is a lower alkyl group;
or
R₁ represents a monocyclic or polycyclic heterocyclic group consisting of from 3 to 15 atoms, at least one of which is selected from the group consisting of N, O, P and —S—(O)₀₋₂; said heterocyclic group optionally by one or more substituents selected from the group consisting of halo, oxo, hydroxyl, lower alkyl, substituted lower all, alkoxy, aryl, acyl, aryloxy, carboxyl, substituted aryl, heterocycle, heteroaryl substituted heteroaryl, cycloalkyl, substituted cycloalkyl, —NO₂, and —CN;
R₂ represents hydrogen; halogen; substituted or unsubstituted lower alkyl or alkenyl group; lower haloalkyl or alkenyl; cyano; acetoamido; lower alkoxy; lower alkylamino; NR^{d}R^{e} where R^{d} and R^{e} are independently hydrogen, lower alkyl, phenyl or phenyl substituted by lower alkyl, lower alkoxy, halogen or haloalkyl or alkoxyl; —SR^{f} where R^{f} is hydrogen, lower alkyl, lower alkanoyl, benzoyl or phenyl;
W represents the group —OCH₂—, —NHCH₂—, —SCH₂— or —NH(C═O)—;
R₃, R₄ and R₅ represent independently a hydrogen, lower alkyl or lower alkenyl, branched or unbranched C₁-C₁₂ alkanoyl, benzoyl or benzoyl substituted by lower alkyl, lower alkoxy, halogen, or R₄ and R₅ form together a 5-membered ring optionally substituted by a lower alkyl or alkenyl; R₃ further represents independently a phosphate, hydrogen or dihydrogen phosphate, or an alkali metal or ammonium or dialkali or diammonium said thereof; or when R₁ represents said monocyclic or polycyclic heterocyclic group, then R₄ and R₅ form together a 5-membered ring optionally substituted by a lower alkyl or alkenyl or R₃, R₄ and R₅ represent each a group of the formula —C(O)R₇ wherein R₇ may be the same or different and is selected from the group consisting of C₁₋₁₅ all, C₂₋₁₅ alkenyl, C₂₋₁₅ alkynyl, heterocyclyl, aryl, and heteroaryl, or which alkyl, alkenyl, alkynyl, aryl, heterocyclyl and heteroaryl are optionally substituted with 1 to 3 substituents independently selected from the group of halo, alkyl —NO₂, heterocyclyl, aryl, heteroaryl, —CF₃, —CN, —OR²⁰, —SR²⁰, —N(R²⁰)₂, —S(O)R²², —SO₂R²², —SO₂N(R²⁰)₂, —SO₂NR²⁰C(O)R²², —SO₂NR²⁰CO₂R²², —SO₂NR²⁰C(O)N(R²⁰)₂, —N(R²⁰)₂, —NC(O)R²², —NR²⁰C(O)R²²—NR²⁰CO₂R²², —NR²⁰C(O)N(R²⁰)₂, —NR²⁰C(NR²⁰)NHR²³, —C(O)R²⁰, —CO₂R²⁰, —CON(R²⁰)₂, —CONR²⁰SO₂R²², —CONR²⁰SO₂R²², —SO₂NR²⁰CO₂R²², —OC(O)NR²⁰SO₂R²², —OC(O)R²⁰, —C(O)OCH₂OC(O)R²⁰, and —OC(O)N(R²⁰)₂; wherein
R²⁰ is a member selected from the group consisting of C₁₋₁₅ alkyl, C₂₋₁₅ alkynyl, C₂₋₁₅ alkynyl, heterocyclyl, aryl and heteroaryl, which all, alkenyl, alkynyl, heterocyclyl, aryl and heteroaryl arc optionally substituted with 1 to 3 substituents independently selected from halo, alkyl, mono or dialkylamino, alkyl-, aryl- or heteroaryl amide, CN, —OC₁₋₆ alkyl, —CF₃, aryl and heteroaryl; and
R²² is a member selected from the group consisting of C₁₋₁₅ alkyl, C₂₋₁₅ alkynyl, C₂₋₁₅ alkynyl, heterocyclyl, aryl and heteroaryl, which alkyl, alkenyl, alkynyl, heterocyclyl, aryl and heteroaryl are optionally substituted with 1 to 3 substituents independently selected from halo, alkyl, mono or dialkylamino, alkyl-, aryl- or heteroaryl amide, CN, —OC₁₋₆alkyl, —CF₃, aryl and heteroaryl;
R₆ represents a hydrogen or halogen atom; or one of the substituents R₁ to R₆ is a sulfohydrocarbon radical of the formula R⁸—SO₃—R^{h}—, wherein R^{g} represents a group selected from C₁-C₁₀ aliphatic, phenyl and lower alkyl substituted aromatic group which may be substituted or unsubstituted and R^{h} represents a monovalent cation, and the non-sulfur containing substituents being as defined above;
and isomers, diastereomers, pharmaceutically acceptable salts or solvates of said compound.

6. A method according to claim 5, wherein said compound is selected from the group consisting of N⁶-cyclopentyl adenosine (CPA), 2-chloro-CPA (CCPA), and N⁶-cyclohexyl adenosine (CHA).

7. A method according to claim 5, wherein R1 is selected from the group consisting of 3-tetrahydrofuranyl, 3-tetrahydrothiofuranyl, 4-pyranyl and 4-thiopyranyl.

8. A method according to claim 7, wherein said compound is N—(3(R)—tetrahydrofuranyl)6-aminopurine riboside.

9. A method for preventing reduction in level of leukocytes in a subject as a result of a treatment comprising administering to the individual an effective amount of an adenosine A1 receptor agonist.

10. A method according to claim 9, wherein said leukocytes are neutrophils.

11. A method according to claim 9, wherein sad treatment is a drug treatment.

12. A method according to claim 11, wherein said drug is an anti-cancer chemotherapeutic drug or a neuroleptic drug.

13. A method according to claim 9, wherein said adenosine A1 receptor agonist is a compound of general formula (I):— 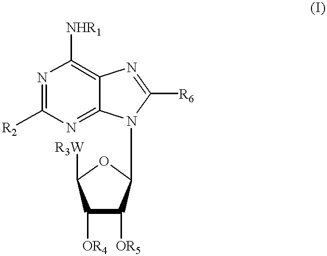
wherein
R₁ represents a lower alkyl substituted or unsubstituted cycloalkyl, a hydroxyl or hydroxyalkyl; a phenyl, lower alkyl phenyl or anilide, all optionally substituted by one or more substituents; —SOR^{c}, —SO₂R^{c}, —SO₃H, —SO₂NR^{a}R^{b}, —OR^{a}, —SR^{a}, NHSO₂R^{c}, —NHCOR^{a}, —NR^{a}R^{b}, or —NHR^{a}CO₂R^{b}; wherein
R^{a} and R^{b} represent independently a hydrogen, lower alkyl, alkanoyl, amine, phenyl or naphthyl, the alkyl group optionally being substituted with a substituted or unsubstituted phenyl or phenoxy group; or when R₁ represents —NR^{a}R^{b}, said R^{a} and R^{b} form together wit the nitrogen atom a 5- or 6-membered heterocyclic ring optionally containing a second heteroatom selected from oxygen or nitrogen, which second nitrogen heteroatom may optionally be further substituted by hydrogen or lower alkyl; or —NR^{a}R^{b} is a group of general formulae (II) or (III):— [chemical structure]
wherein
n is an integer from 1 to 4;
Z is hydrogen, lower alkyl or hydroxyl;
Y is hydrogen, lower alkyl, or OR′ where R′ is hydrogen, lower alkyl or lower alkanoyl;
A is a bond or a lower alkylene, preferably, C₁-C₄ alkenyl;
X and X′ are each independently hydrogen, lower alkyl, lower alkoxy, hydroxy; lower alkanoyl, nitro, haloalkyl such as trifluoromethyl, halogen, amino, mono or di-lower alkyl amino, or when X and X′ are taken together a methylenedioxy group;
R^{c} represents a lower alkyl;
or
R₁ represents an epoxide substituent of general formulae (IVa) or (IVb): 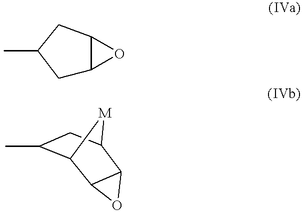
wherein M is a lower alkyl group; or
R₁ represents a monocyclic or polycyclic heterocyclic group consisting of from 3 to 15 atoms, at least one of which is selected from the group consisting of N, O, P and S—(O)₀₋₂; said heterocyclic group optionally substituted by one or more substituents selected from the group consisting of halo, oxo, hydroxyl, lower alkyl substituted lower alkyl, alkoxy, aryl, acyl, aryloxy, carboxyl, substituted aryl, heterocycle, heteroaryl, substituted heteroaryl, cycloalkyl, substituted cycloalkyl, —NO₂, and —CN;
R₂ represents hydrogen; halogen; substituted or unsubstituted lower alkyl or alkenyl group; lower haloalkyl or alkenyl; cyano; acetoamido; lower alkoxy; lower alkylamino; NR^{d}R^{e} where R^{d} and R^{e} are independently hydrogen, lower alkyl, phenyl or phenyl substituted by lower alkyl, lower alkoxy, halogen or haloalkyl or alkoxyl; —SR^{f} where R^{f} is hydrogen, lower alkyl, lower alkanoyl, benzoyl or phenyl;
W represents the group —OCH₂—, —NHCH₂—, —SCH₂—or —NH(C═O)—;
R₃, R₄ and R₅ represent independently a hydrogen, lower alkyl or lower alkenyl, branched or unbranched C₁-C₁₂ alkanoyl, benzoyl or benzoyl substituted by lower alkyl, lower alkoxy, halogen, or R₄ and R₅ form together a 5-membered ring optionally substituted by a lower alkyl or alkenyl; R₃ further represents independently a phosphate, hydrogen or dihydrogen phosphate, or an alkali metal or ammonium or dialkali or diammonium said thereof; or when R₁ represents said monocyclic or polycyclic heterocyclic group, then R₄ and R₅ form together a 5-membered ring optionally substituted by a lower alkyl or alkenyl or R₃, R₄ and R₅ represent each a group of the formula —C(O)R₇ wherein R₇ may be die same or different and is selected from the group consisting of C₁₋₁₅ alkyl, C₂₋₁₅ alkenyl, C₂₋₁₅ alkynyl, heterocyclyl, aryl, and heteroaryl, or which alkyl, alkenyl, alkynyl, aryl, heterocyclyl and heteroaryl are optionally substituted with 1 to 3 substituents independently selected from the group of halo, alkyl —NO₂, heterocyclyl, aryl, heteroaryl, —CF₃, —CN, —OR²⁰, —SR²⁰, —N(R²⁰)₂, —S(O)R²², —SO₂R²², —SO₂N(R²⁰)₂, —SO₂NR²⁰C(O)R²², —SO₂NR²⁰CO₂R²², —SO₂NR²⁰C(O)N(R²⁰)₂, —N(R²⁰)₂, —NC(O)R²², —NR²⁰C(O)R²², —NR²⁰CO₂R²², —NR²⁰C(O)N(R²⁰)₂, —NR²⁰C(NR²⁰)NHR²³, —C(O)R²⁰, —CO₂R²⁰, —CON(R²⁰)₂, —CONR²⁰SO₂R²², —NR²⁰SO₂R²², —SO₂NR²⁰CO₂R²², —OC(O)NR²⁰SO₂R²², —OC(O)R²⁰, —C(O)OCH₂OC(O)R²⁰, and —OC(O)N(R²⁰)₂; wherein
R²⁰ is a member selected from the group consisting of C₁₋₁₅ alkyl, C₂₋₁₅ alkynyl, C₂₋₁₅ alkynyl, heterocyclyl, aryl and heteroaryl, which alkyl, alkenyl, alkynyl, heterocyclyl, aryl and heteroaryl are optionally substituted with 1 to 3 substituents independently selected from halo, alkyl, mono or dialkylamino, alkyl- aryl- or heteroaryl amide, CN, —OC₁₋₆alkyl, —CF₃, aryl and heteroaryl; and
R²² is a member selected from the group consisting of C₁₋₁₅ alkyl, C₂₋₁₅ alkynyl, C₂₋₁₅ alkynyl, heterocyclyl, aryl and heteroaryl, which alkyl, alkenyl, alkyl, heterocyclyl, aryl and heteroaryl are optionally substituted with 1 to 3 substituents independently selected from halo, alkyl, mono or dialkylamino, alkyl-, aryl- or heteroaryl amide, CN, —OC₁₋₆alkyl, —CF₃, aryl and heteroaryl;
R₆ represents a hydrogen or halogen atom; or one of the substituents R₁ to R₆ is a sulfohydrocarbon radical of the formula R^{g}—SO₃-R^{h}—, wherein R^{g} represents a group selected from C₁-C₁₀ aliphatic, phenyl and lower alkyl substituted aromatic group which may be substituted or unsubstituted and R^{h} represents a monovalent cation, and the non-sulfur containing substituents being as defined above;
and isomers, diastereomers, pharmaceutically acceptable salts or solvates of said compound.

14. A method according to claim 13, wherein said compound is selected from the group consisting of N⁶-cyclopentyl adenosine (CPA), 2-chloro-CPA (CCPA), and N⁶-cyclohexyl adenosine (CHA).

15. A method according to claim 13, wherein R1 is selected from the group consisting of 3-tetrahydrofuranyl, 3-tetrahydrothiofuranyl, 4-pyranyl and 4-thiopyranyl.

16. A method according to claim 15, wherein said compound is N—(3(R)— tetrahydrofuranyl)-6-aminopurine riboside.

17. A method of treatment of an individual comprising administering to the subject a therapeutic drug in combination with an adenosine A1 receptor agonist.

18. A method according to claim 17, wherein the adenosine A1 receptor agonist is administered prior or during a course of administration of the therapeutic drug.

19. A method according to claim 17, wherein said adenosine A1 receptor agonist is a compound of general formula (I):— 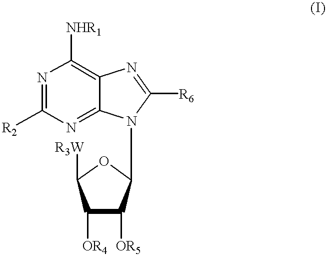
wherein
R₁ represents a lower alkyl, substituted or unsubstituted cycloalkyl; a hydroxyl or hydroxyalkyl; a phenyl, lower alkyl phenyl or anilide, all optionally substituted by one or more substituents, —SOR^{c}, —SO₂R^{c}, —SO₃H, —So₂NR^{a}R^{b}, —OR^{a}, —SR^{a}, —NHSO₂R^{c}, —NHCOR^{a}, —NR^{a}R^{b}, or —NHR^{a}CO₂R^{b}; wherein
R^{a} and R^{b} represent independently a hydrogen lower alkyl, alkanoyl, amine, phenyl or naphthyl, the alkyl group optionally being substituted with a substituted or unsubstituted phenyl or phenoxy group; or when R₁ represents —NR^{a}R^{b}, said R^{a} and R^{b} form together with the nitrogen atom a 5- or 6- membered heterocyclic ring optionally containing a second heteroatom selected from oxygen or nitrogen, which second nitrogen heteroatom may optionally be further substituted by hydrogen or lower alkyl; or —NR^{a}R^{b} is a group of general formulae (II) or (III):— [chemical structure]
wherein
n is an integer from 1 to 4;
Z is hydrogen, lower alkyl or hydroxyl;
Y is hydrogen, lower alkyl, or OR′ where R′ is hydrogen, lower alkyl or—lower alkanoyl;
A is a bond or a lower alkylene, preferably; C₁-C₄ alkenyl;
X and X′ are each independently hydrogen, lower alkyl, lower alkoxy, hydroxy, lower alkanoyl, nitro, haloalkyl such as trifluoromethyl halogen, amino, mono- or di-lower alkyl amino, or when X and X′ are taken together a methylenedioxy group;
R^{c} represents a lower alkyl; or
R₁ represents an epoxide substituent of general formulae (IVa) or (IVb): 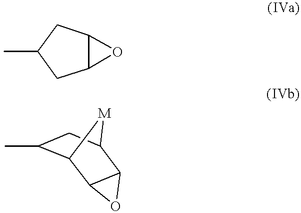
wherein M is a lower alkyl group; or
R₁ represents a monocyclic or polycyclic heterocyclic group consisting of from 3 to 15 atoms, at least one of which is selected from the group consisting of N, O, P and S—(O)₀₋₂; said heterocyclic group optionally substituted by one or more substituents selected from the group consisting of halo, oxo, hydroxyl, lower alkyl, substituted lower alkyl, alkoxy, aryl, acyl, aryloxy, carboxyl, substituted aryl, heterocycle, heteroaryl, substituted heteroaryl, cycloalkyl, substituted cycloalkyl,—NO₂, and —CN
R₂ represents hydrogen; halogen; substituted or unsubstituted lower alkyl or alkenyl group; lower haloalkyl or alkenyl; cyano; acetoamido; lower alkoxy; lower alkylamino NR^{d}R^{e} where R^{d} and R^{e} are independently hydrogen, lower alkyl, phenyl or phenyl substituted by lower alkyl, lower alkoxy, halogen or haloalkyl or alkoxyl; —SR^{f} where R^{f} is hydrogen, lower alkyl, lower alkanoyl, benzoyl or phenyl;
W represents the group —OCH₂—, —NHCH₂—, —SCH₂— or —NH(C═O)—;
R₃,R₄ and R₅ represent independently a hydrogen, lower alkyl or lower alkenyl, branched or unbranched C₁-C₁₂ alkanoyl, benzoyl or benzoyl substituted by lower alkyl, lower alkoxy, halogen, or R₄ and R₅ form together a 5-membered ring optionally substituted by a lower alkyl or alkenyl; R₃ further represents independently a phosphate, hydrogen or dihydrogen phosphate, or an alkali metal or ammonium or dialkali or diammonium said thereof; or when R₁ represents said monocyclic or polycyclic heterocyclic group, then R₄ and R₅ form together a 5-membered ring optionally substituted by a lower alkyl or alkenyl or R₃, R₄ and R₅ represent each a group of the formula —C(O)R₇ wherein R₇ may be the same or different and is selected from the group consisting of C₁₋₁₅ alkyl, C₂₋₁₅ alkenyl, C₂₋₁₅ alkynyl, heterocyclyl, aryl, and heteroaryl, or which alkyl, alkenyl, alkynyl, aryl, heterocyclyl and heteroaryl are optionally substituted with 1 to 3 substituents independently selected from the group of halo, alkyl —NO₂, heterocyclyl, aryl, heteroaryl, —CF₃, —CN, —OR²⁰, —SR²⁰, —N(R²⁰)₂, —S(O)R²²—SO₂R²², —SO₂N(R²⁰)₂, —SO₂NR²⁰C(O)R²², —SO₂NR²⁰CO₂R²², —SO₂NR²⁰C(O)N(R²⁰)₂, —N(R²⁰)₂, —NC(O)R²², —NR²⁰C(O)R²², —NR²⁰CO₂R²², —NR²⁰C(O)N(R²⁰)₂, —NR²⁰C(NR²⁰)NHR²³, —C(O)R²⁰, —CO₂R²⁰, —CON(R²⁰)₂, —CONR²⁰So₂R²², —NR²⁰SO₂R²², —SO₂NR²⁰CO₂R²², —OC(O)NR²⁰SO₂R²², —OC(O)R²⁰, —C(O)OCH₂OC(O)R²⁰, and —OC(O)N(R²⁰)₂; wherein
R²⁰ is a member selected from the group consisting of C₁₋₁₅ alkyl, C₂₋₁₅ alkynyl, C₂₋₁₅ alkynyl, heterocyclyl, aryl and heteroaryl, which alkyl, alkenyl, alkynyl, heterocyclyl, aryl and heteroaryl are optionally substituted with 1 to 3 substituents independently selected from halo, alkyl, mono or dialkylamino, alkyl-, aryl- or heteroaryl amide, CN —OC₁₋₆alkyl, —CF₃, aryl and heteroaryl, and
R²² is a member selected from the group consisting of C₁₋₁₅ alkyl, C₂₋₁₅ alkyl, C₂₋₁₅ alkynyl, heterocyclyl, aryl and heteroaryl, which alkyl), alkenyl, alkynyl, heterocyclyl, aryl and heteroaryl are optionally substituted with 1 to 3 substituents independently selected from halo, alkyl, mono or dialkylamino, alkyl-, aryl- or heteroaryl amide, CN, —OC₁₋₆alkyl, —CF₃, aryl and heteroaryl;
R₆ represents a hydrogen or halogen atom; or
one of the substituents R₁ to R₆ is a sulfohydrocarbon radical of the formula R⁶—SO₃-R^{b}—, wherein R^{g} represents a group selected from C₁-C₁₀ aliphatic, phenyl and lower alkyl substituted aromatic group which may be substituted or unsubstituted and R^{h} represents a monovalent cation, and the non-sulfur containing substituents being as defined above;
and isomers, diastereomers, pharmaceutically acceptable salts or solvates of said compound.

20. The method according to claim 19, wherein said compound is selected from the group consisting of N⁶-cyclopentyl adenosine (CPA), 2-chloro-CPA (CCPA), and NC-cyclohexyl adenosine (CHA).

21. A method according to claim 19, wherein R1 is selected from the group consisting of 3-tetrahydrofuranyl, 3-tetrahydrothiofuranyl, 4-pyranyl and 4-thiopyranyl.

22. A method according to claim 21, wherein said compound is N-(3(R)-tetrahydrofuranyl)-6-aminopurine riboside.
